(19) United States
(12) Patent Application Publication
(10) Pub. No.: US 2024/0353434 A1
(43) Pub. Date: Oct. 24, 2024

(54) METHOD FOR SIMULTANEOUS AND EFFICIENT DETECTION OF RESIDUES OF 36 ANTIDEPRESSANTS IN SLUDGES

(71) Applicant: Institute of Microbiology, Guangdong Academy of Sciences (Guangdong Detection Center of Microbiology, Guangzhou (CN)

(72) Inventors: Meiying XU, Guangzhou (CN); Jinna ZHANG, Guangzhou (CN); Ariguo WANG, Guangzhou (CN); Sihua LAI, Guangzhou (CN)

(73) Assignee: Institute of Microbiology, Guangdong Academy of Sciences (Guangdong Detection Center of Microbiology, Guangzhou (CN)

(21) Appl. No.: 18/238,544

(22) Filed: Aug. 28, 2023

(30) Foreign Application Priority Data
Apr. 20, 2023 (CN) 202310422822.8

(51) Int. Cl. G01N 33/94; G01N 1/34; G01N 33/68

(52) U.S. Cl. CPC G01N 33/9466; G01N 1/34; G01N 33/6851

(57) ABSTRACT

A method for simultaneous and efficient detection of residues of 36 antidepressants in sludge includes extraction of target drugs, purification of an extraction solution, detection by the technique of ultra-performance liquid chromatography-tandem mass spectrometry, and qualitative and quantitative analysis of the antidepressants. Based on the detection technique of ultra-performance liquid chromatography-tandem mass spectrometry, by using UPLC/QTOF-MS in combination with pretreatment methods. The method solves the problems such as absence of detection methods for some antidepressants in existing detection methods, high time consumption of sample pretreatment experiments, high detection costs, and low resolution of detection methods. According to the method, the recovery rate of antidepressants is 63.13-115.66%, and the limit of detection of the method is 0.14-13.47 ng/g; only 0.5 g of dry sludge sample is required. The experimental process is simplified, the introduced random errors are reduced, and the time and detection cost consumed by the experiment are greatly reduced.

### CROSS REFERENCE TO THE RELATED APPLICATIONS

This application is based upon and claims priority to Chinese Patent Application No. 202310422822.8, filed on Apr. 20, 2023, the entire contents of which are incorporated herein by reference.

### TECHNICAL FIELD

The present invention belongs to the technical field of detection of environmental trace pollutants, and in particular, relates to a method for simultaneous and efficient detection of residues of 36 antidepressants in sludge.

### BACKGROUND

Antidepressants are a kind of drugs for the treatment of major depressive disorder, anxiety, obsessive-compulsive disorder, drinking and eating disorders, chronic pain and neuropathic pain, and also for use in adjuvant treatment of symptoms of dysmenorrhea, snoring, migraine, attention deficit hyperactivity disorder, drug abuse, and insomnia. According to statistics, the production and consumption of antidepressants have climbed rapidly due to the surge in the number of people with mental disorders. The antidepressants and their metabolites, which have not been transformed and absorbed by the human body, enter the environment along with excreta, leading to a continuous increase in the emission of these drugs into the environment. Long-term exposure of environmental organisms to environmental media containing antidepressants could lead to bioaccumulation, which in turn causes abnormal behaviors and reproductive toxicity of organisms, and might result in ecological and environmental risks.

As a main storage and treatment system for municipal sewage and town sewage, wastewater treatment plants (WWTPs) are key barriers for blocking the discharge of emerging contaminants into the environment. According to existing studies, sludge adsorption is the main mechanism for the removal of antidepressant drugs in WWTPs. Hence, there are different degrees of residues of antidepressants and their metabolites in the sludge. After concentration, dehydration and stabilization, the remaining sludge is directly landfilled or used in building materials, soil improvement, etc. The residual antidepressants therein, however, may transport to the soil or groundwater, leading to further contamination. To assess the degree of risk of the further contamination, it is necessary to comprehensively understand the residue levels of antidepressants in the sludge. Therefore, it is essential to develop an analysis method for simultaneous determination of residues of multiple antidepressants in the sludge.

### SUMMARY

It is an object of the present invention to provide a method capable of simultaneously detecting multiple antidepressants in sludge, which can rapidly and efficiently detect 36 antidepressants in the sludge. The method is characterized by a low limit of detection, high sensitivity, and a high detection speed.

The technical solution of the present invention is as follows.

A method for simultaneous and efficient detection of residues of 36 antidepressants in sludge includes the following steps:
step 1, extraction of target drugs: weighing a sludge sample, and then performing solvent extraction to obtain an extraction solution containing the antidepressants;
step 2, purification of the extraction solution: purifying the extraction solution;
step 3, detection of the purified extraction solution by ultra-performance liquid chromatography-tandem mass spectrometry; and
step 4: qualitative and quantitative analysis of the antidepressants;
wherein the 36 antidepressants include agomelatine, alprazolam, amitriptyline, bupropion, buspirone, carbamazepine, chlordiazepoxide, clobazam, clonazepam, clozapine, desvenlafaxine, diazepam, doxepin, duloxetine, escitalopram, estazolam, flunitrazepam, fluoxetine, flurazepam, imipramine, lamotrigine, mianserin, midazolam, mirtazapine, moclobemide, nitrazepam, nordazepam, nortriptyline, prazepam, quetiapine, reboxetine, ropivacaine, sertraline, tandospirone, trazodone, and venlafaxine;
purifying the extraction solution includes: passing the extraction solution collected in step 1 through pass-through EMR-LPD cartridges containing Carbon S, collecting a purified effluent, drying the effluent with nitrogen at room temperature, redissolving the dried effluent in methanol, mixing a resultant evenly, and filtering a resulting mixture to collect a filtrate, which is preserved in the dark for later on-instrument detection; and
performing the solvent extraction to obtain the extraction solution containing the antidepressants includes the following steps: freeze-drying the sludge samples, grinding and sieving the freeze-dried sludge, weighing the sludge sample, successively adding an inorganic extractant and an organic extractant to the sample, mixing a resultant evenly, and then performing ultrasonic and centrifugal extraction to collect a supernatant, wherein the inorganic extractant is an aqueous ammonia solution with pH of 11, and the organic extractant is one or more of methanol, ethyl acetate and n-hexane.

Preferably, detection conditions for the technique of ultra-performance liquid chromatography-tandem mass spectrometry include:
conditions for liquid chromatography: ACQUITY UPLC BEH C18 columns, 2.1 mm×100 mm, column temperature: 40° C., flow rate: 0.35 mL/min, injection volume: 2 μL, mobile phase A: 0.1% formic acid in Milli-Q water, and mobile phase B: acetonitrile; and gradient elution conditions: 0-28 min, 90/6-10% of A; 28-30 min, 10%-90% of A, for 2 min, with the mobile phases A and B in an eluent being 100% in total in each stage; and
detection conditions for mass spectrometry: with an electrospray ion source and under a positive ion mode, simultaneous acquisition of MS data of high- and low-collision energy by using an MS^{E} scanning mode in a single injection, wherein compound precursor ions are collected in low-energy channels and fragment ions are collected in high-energy channels; collision voltage: low energy: 6 V, high energy: 10 V-40 V; scanning range: m/z: 50-1000, ion source temperature: 120° C.; capillary voltage: 2.5 kV; cone voltage 40 V; cone gas flow rate: 50 L/Hr; and drying gas: nitrogen, temperature: 500° C., and flow rate: 600 L/Hr.

Preferably, performing the solvent extraction to obtain the extraction solution containing the antidepressants includes the following specific steps:
(1) freeze-drying: freeze-drying the sludge sample for 48 hours by using a freeze dryer at −60° C. under vacuum conditions, grinding the freeze-dried sludge sample, sieving the ground sludge sample through a 100-mesh sieve to obtain homogeneous sludge particles, and cold-preserving the sludge particles at −20° C.; and
(2) ultrasonic and centrifugal extraction: weighing 0.5 g of the sludge sample, placing the sludge sample in a 50 mL centrifuge tube, and performing the following steps:
A. adding 4 mL of an aqueous ammonia solution, which is adjusted to pH 11 with HPLC-grade 25% ammonium hydroxide, and 1 mL of methanol to the sample tube and vortexing the sample for 5 min;
B. adding 5 mL of ethyl acetate to the sample tube, performing vortexing for thorough mixing, then placing the sample tube in an ultrasonic bath for extracting for 10 min, then centrifuging for 5 min at 4500 rpm to collect a supernatant, repeating the steps of ultrasonic extraction and centrifugation once, and combining the supernatants; and
C. adding 4 mL of n-hexane to the sample tube, performing vortexing for thorough mixing, then placing the sample tube in an ultrasonic bath for extracting for 10 min, then centrifuging for 5 min at 4500 rpm, collecting a supernatant, and combining the supernatant to obtain the extraction solution.

Preferably, the qualitative and quantitative analysis of the antidepressants includes: performing qualitative analysis by the exact mass, retention time, and characteristic ions; and performing quantitative analysis by an external standard method.

Preferably, the qualitative and quantitative analysis specifically includes:
a qualitative method, including: with qualitative analysis on target drugs performed in a UNIFI scientific information system, before the qualitative analysis, drawing chemical structures of target antidepressants to construct a target drug database, which is imported into the UNIFI scientific information system; then, establishing an analysis method, wherein after a desired ionization mode is selected, the target drug database is imported as a list of target components for the analysis method, and corresponding screening parameters are set, specifically with an exact mass error of less than 3 ppm for the compound precursor ions, and with an exact mass error of less than 10 ppm for the fragment ions; acquiring MS^{E} data of 100 μg/L individual target antidepressant standards by using a detection method in step 3, then analyzing the data in the UNIFI scientific information system, and performing matching one by one to obtain measured exact masses under an instrument method, retention times, and mass spectrum information under high- and low-energy for the individual antidepressants; and assigning the information obtained by using the standards to the individual compounds in the database, and establishing a qualitative method for the sample by using the database containing the measured exact masses, retention times, and fragment ion information of the individual antidepressants, wherein a matching principle for the target antidepressants in an actual sample is as follows: desired fragment ions can be matched with the exact mass error of less than 2 ppm and a retention time error of less than 0.1 min; and
a quantitative method, including: establishing a quantitative analysis method based on the external standard method in UNIFI, and setting default concentration levels of a standard curve in the list of target components for the qualitative method, which specifically are 1, 5, 10, 20, 50, 100 and 200 ng/g mixed standard working solutions, with a linear fitting mode for the standard curve; acquiring MS^{E} data of mixed standard working solutions of a series of these concentrations, importing the MS^{E} data into the UNIFI, establishing analysis with the established analysis method, and after the data analysis is completed, automatically providing a standard working curve by software, adding MS^{E} data of an actual environmental sludge sample to the quantitative analysis of the standards, and performing analysis to subsequently obtain concentrations of the target antidepressants in the sample.

The present invention further provides use of the method described above in detection of residues of 36 antidepressants in sludge, wherein the 36 antidepressants include: agomelatine, alprazolam, amitriptyline, bupropion, buspirone, carbamazepine, chlordiazepoxide, clobazam, clonazepam, clozapine, desvenlafaxine, diazepam, doxepin, duloxetine, escitalopram, estazolam, flunitrazepam, fluoxetine, flurazepam, imipramine, lamotrigine, mianserin, midazolam, mirtazapine, moclobemide, nitrazepam, nordazepam, nortriptyline, prazepam, quetiapine, reboxetine, ropivacaine, sertraline, tandospirone, trazodone, and venlafaxine.

The antidepressants include one or more of SARIs antidepressants, SNRIs antidepressants, SSRIs antidepressants, TCAs antidepressants, NRIs antidepressants, NaSSA antidepressants, MAOIs antidepressants, BZDs antidepressants, P5-HAs antidepressants, lamotrigine, agomelatine and ropivacaine. The SARIs antidepressants include trazodone; the SNRIs antidepressants include one or more of duloxetine, desvenlafaxine, and venlafaxine; the SSRIs antidepressants include one or more of escitalopram, fluoxetine and sertraline; the TCAs antidepressants include one or more of amitriptyline, imipramine, doxepin, carbamazepine and nortriptyline; the NRIs antidepressants include one or more of bupropion and reboxetine; the NaSSAs antidepressants include one or more of mianserin and mirtazapine; the MAOIs antidepressants include moclobemide; the BZDs antidepressants include one or more of alprazolam, estazolam, diazepam, flurazepam, flunitrazepam, chlordiazepoxide, clozapine, flunitrazepam, midazolam, prazepam, nordazepam, nitrazepam and clobazam; and the P5-HAs antidepressants include one or more of buspirone and tandospirone.

The present invention has the following beneficial effects. A quantitative analysis method for simultaneous detection of trace amounts of 36 antidepressants in sludge is provided by means of an ultra-performance liquid chromatography-tandem quadrupole time-of-flight mass spectrometer (UPLC/QTOF-MS) in combination with pretreatment methods such as solid-liquid ultrasonic and centrifugal extraction and pass-through cartridge purification. The method has the following advantages:
(1) the method provided by the present invention shows a high recovery rate (63.13-115.66%) for antidepressants and a low limit of detection (0.14-13.47 ng/g), and could simultaneously detect up to 36 target antidepressants, with fewer operation steps, and high efficiency;
(2) instead of the conventional method including steps such as rotary evaporation and subsequent SPE solid-phase extraction, the present invention uses the pass-through EMR cartridges to purify the extraction solution from ultrasonic extraction, which simplifies the experimental process, greatly improves the simplicity of experimental operation, greatly reduces the experimental time consumption, and simultaneously helps to reduce the introduced random error of the experiment;
(3) the present invention requires a small amount of sludge samples for detection, and only 0.5 g of dry sludge is needed to achieve the effective determination of 36 antidepressants;
(4) in the process of the present invention, the use of expensive SPE extraction cartridges such as HLB is avoided, and meanwhile, with the external standard method for quantification of the target substance, the use of expensive internal standards is avoided, which greatly reduces the cost of analysis and detection; and
(5) the liquid-phase separation method used in the present invention adopts a general gradient, is simple to set up, has a wide range of applications, and can be used for detection and screening of a wide range of compounds, which greatly reduces the difficulty in detection method development and shortens the time consumed for method development; and meanwhile, the sample is collected by using an MS^{E} scanning mode, such that massive compound information can be obtained, thereby providing more abundant raw data for subsequent in-depth data mining.

### BRIEF DESCRIPTION OF THE DRAWINGS

FIG. 1 shows extracted ion chromatogram of 18 target antidepressant standards, including agomelatine, chlordiazepoxide, doxepin, alprazolam, clobazam, duloxetine, amitriptyline, clonazepam, escitalopram, bupropion, clozapine, estazolam, buspirone, desvenlafaxine, flunitrazepam, carbamazepine, diazepam, and fluoxetine; and

FIG. 2 shows extracted ion chromatogram of another 18 target antidepressant standards, including flurazepam, nitrazepam, ropivacaine, imipramine, nordazepam, sertraline, lamotrigine, nortriptyline, tandospirone, mianserin, prazepam, trazodone, midazolam, quetiapine, venlafaxine, mirtazapine, reboxetine, and moclobemide.

### DETAILED DESCRIPTION OF THE EMBODIMENTS

The present invention is further described in detail by the embodiments below. Experimental methods used in the embodiments are conventional methods, unless otherwise specified. The materials, reagents, methods, and instruments used, unless otherwise specified, are conventional materials, reagents, methods, and instruments in the art, which are commercially available for those skilled in the art.

The following embodiments are intended to further illustrate the present invention, rather than limiting the present invention.

### Embodiment 1

### (1) Collection and Preservation of Samples

Sludge from a sewage treatment plant (WWTP) in Guangzhou or river sediment was collected, preserved at 4° C., transported to the laboratory, frozen at −20° C. for 12 hours, and then freeze-dried for 48 hours using a freeze dryer at −60° C. under vacuum conditions. The freeze-dried sludge was ground and sieved through a 100-mesh sieve to obtain homogeneous sludge particles, which were cold-preserved at −20° C.

### (2) Formulation and Preparation of Inorganic and Organic Extraction Solutions for Antidepressants in Sludge

The extraction solutions included: an pH 11 aqueous ammonia solution (adjusted by HPLC-grade 25% ammonium hydroxide), methanol, ethyl acetate, and n-hexane;

### (3) Extraction of Target Drugs

0.5 g (accurate to 0.0001 g) of a sludge sample was weighed and placed in a 50 mL centrifuge tube, and the following steps were performed: adding 1 mL of methanol and 4 mL of aqueous ammonia solution to the sample tube, and performing vortexing for 5 min for thorough mixing; adding 5 mL of ethyl acetate to the sample tube and performing vortexing for thorough mixing; then performing ultrasonic extraction (with the power of 800 W, the same below) for 10 minutes and performing centrifugation for 5 min at 4500 rpm and collecting a supernatant; repeating this step once and then combining the supernatants; adding 4 mL of n-hexane to the sample tube, performing vortexing for thorough mixing, performing ultrasonic extraction for 10 min and performing centrifugation for 5 min at 4500 rpm; and collecting a supernatant, and combing all supernatants to obtain a extraction solution.

### (4) Purification of Extraction Solution

The extraction solution of target substances was purified by pass-through EMR-LPD cartridges (with Carbon S, 6 mL, Agilent); the extraction solution was loaded into the purification cartridges at the speed of 0.5 mL/min under the action of gravity flow; the purified effluent was collected, and then the EMR-LPD cartridges were rinsed and purified using 2 mL of methanol, the rinsing solution was extracted and collected under a negative pressure by using a vacuum pump; and the rinsing solution and the purified effluent were combined.

### (5) Concentration of Antidepressants

The rinsing solution and the purified effluent were combined and then dried with a stable nitrogen gas stream at room temperature; the extract was redissolved in 1 mL of methanol, vortexed evenly and filtered with a 0.22 μm organic filter membrane. The filtrate was collected and placed in a 2 mL brown LC vial, and preserved in the dark at −20° C. for later on-instrument detection.

### (6) Detection of Sample by UPLC/QTOF-MS

The sample was detected using the ultra-performance liquid chromatography-tandem quadrupole time-of-flight mass spectrometer (UPLC/QTOF-MS, Waters Corp., Milford, MA, U.S.A.). The liquid-phase separation method was performed using the ACQUITY UPLC BEH C18 columns of 2.1 mm×100 mm, where the column temperature was 40° C.; the flow rate was 0.35 mL/min; the injection volume was 2 μL; the mobile phase A was an formic acid in Milli-Q water with a volume ratio of 1:1000, and the mobile phase B was acetonitrile; and the gradient elution conditions were as follows (described by the volume fraction of mobile phase A): 0-28 min, 90%-10% of A; 28-30 min, 10%-90% of A, for 2 min, with the mobile phases A and B in an eluent being 100% in total in each stage.

The detection conditions for mass spectrometry were as follows: with an electrospray ion source and under a positive ion mode, simultaneous acquisition of MS data of high- and low-collision energy by using an MS^{E} scanning mode in a single injection, wherein compound precursor ions were collected in low-energy channels and fragment ions were collected in high-energy channels; collision voltage: low energy: 6 V, high energy: 10 V-40 V; scanning range: m/z: 50-1000, ion source temperature: 120° C.; capillary voltage: 2.5 kV; cone voltage 40 V; cone gas flow rate: 50 L/Hr; and drying gas: nitrogen, temperature: 500° C., and flow rate: 600 L/Hr.

(7) Data Analysis with UNIFI Scientific Information System

Qualitative analysis: the qualitative analysis was performed on target drugs in a UNIFI scientific information system. Before the qualitative analysis, the chemical structures of 36 target antidepressants were drawn to construct a target drug database, which was imported into the UNIFI scientific information system, so that the database includes key information such as the molecular formulas, structural formulas, and exact masses of target drugs. Next, an analysis method was established, where after a desired ionization mode was selected, the target drug database was imported as a list of target components for the analysis method, and corresponding screening parameters were set, specifically with a exact mass error of less than 3 ppm for the compound precursor ions, and with a exact mass error of less than 10 ppm for the fragment ions. The MS^{E} data of 100 μg/L (indicating the concentration of 100 μg/L for each component) mixed standards of 36 target antidepressants was acquired using the UPLC/QTOF-MS sample detection method in step (6), the data were then analyzed in the UNIFI scientific information system, and matching was performed one by one to obtain measured exact masses under an instrument method, retention times, and mass spectrum information under high- and low-energy for the individual antidepressants. The above information obtained by using the standards was assigned to the individual target compounds in the database, and a qualitative and quantitative method for the sample was established by reusing the database containing the measured exact masses, retention times, and fragment ion information of the individual antidepressants. The matching principle for the target antidepressants in an actual sample was as follows: desired fragment ions could be matched with the exact mass error of less than 2 ppm and a retention time error of less than 0.1 min. The information on the target drugs is shown in Table 1.

TABLE 1
Information on target antidepressants

| | | | Measured | | | |
|---|---|---|---|---|---|---|
| | | | mass-to- | Exact | | |
| | | | charge | mass | Retention | |
| | Chinese | Molecular | ratio of | error | time | |
| Compound | name | formula | precursor | (ppm) | (min) | Adductions |
| Agomelatine | [symbol] | C₁₅H₁₇NO₂ | 244.1336 | 1.4 | 7.64 | +H |
| Alprazolam | [symbol] | C₁₇H₁₃CIN₄ | 309.0902 | 0.3 | 7.88 | +H |
| Amitriptyline | [symbol] | C₂₀H₂₃N | 278.1908 | 1.5 | 8.01 | +H |
| Bupropion | [symbol] | C₁₃H₁₈CINO | 240.1153 | 1.4 | 3.92 | +H |
| Buspirone | [symbol] | C₂₁H₃₁N₅O₂ | 386.255 | −0.2 | 4.62 | +H |
| Carbamazepine | [symbol] | C₁₅H₁₂N₂O | 237.1023 | 0.3 | 6.36 | +H |
| Chlordiazepoxide | [symbol] | C₁₆H₁₄CIN₃O | 300.0902 | 1.4 | 4.06 | +H |
| Clobazam | [symbol] | C₁₆H₁₃CIN₂O₂ | 301.0736 | −0.9 | 8.82 | +H |
| Clonazepam | [symbol] | C₁₅H₁₀CIN₃O₃ | 316.0485 | 0.4 | 7.44 | +H |
| Clozapine | [symbol] | C₁₈H₁₉CIN₄ | 327.1373 | 0.6 | 4.51 | +H |
| Desvenlafaxine | [symbol] | C₁₆H₂₅NO₂ | 264.1961 | 1 | 2.12 | +H |
| Diazepam | [symbol] | C₁₆H₁₃CIN₂O | 285.0792 | 0.8 | 9.28 | +H |
| Doxepin | [symbol] | C₁₉H₂₁NO | 280.1697 | 0.3 | 6.35 | +H |
| Duloxetine | [symbol] | C₁₈H₁₉NOS | 298.1263 | 1 | 7.88 | +H |
| Escitalopram | [symbol] | C₂₀H₂₁FN₂O | 325.1712 | 0.5 | 6.23 | +H |
| Estazolam | [symbol] | C₁₆H₁₁CIN₄ | 295.075 | 1.8 | 7.29 | +H |
| Flunitrazepam | [symbol] | C₁₆H₁₂FN₃O₃ | 314.0941 | 1.9 | 8.03 | +H |
| Fluoxetine | [symbol] | C₁₇H₁₈F₃NO | 310.1413 | 0 | 8.75 | +H |
| Flurazepam | [symbol] | C₂₁H₂₃CIFN₃O | 388.1587 | 0.1 | 6.06 | +H |
| Imipramine | [symbol] | C₁₉H₂₄N₂ | 281.2015 | 1 | 7.59 | +H |
| Lamotrigine | [symbol] | C₉H₇C₁₂N₅ | 256.0155 | 1.4 | 2.35 | +H |
| Mianserin | [symbol] | C₁₈H₂₀N₂ | 265.1704 | 1.7 | 5.95 | +H |
| Midazolam | [symbol] | C₁₈H₁₃CIFN₃ | 326.0856 | 0.3 | 5.62 | +H |
| Mirtazapine | [symbol] | C₁₇H₁₉N₃ | 266.1654 | 0.9 | 2.42 | +H |
| Moclobemide | [symbol] | C₁₃H₁₇CIN₂O₂ | 269.1054 | 1 | 1.94 | +H |
| Nitrazepam | [symbol] | C₁₅H₁IN₃O₃ | 282.0875 | 0.8 | 6.62 | +H |
| Nordazepam | [symbol] | C₁₅H₁₁CIN₂O | 271.0635 | 1 | 7.49 | +H |
| Nortriptyline | [symbol] | C₁₉H₂₁N | 264.1749 | 1 | 7.81 | +H |
| Prazepam | [symbol] | C₁₉H₁₇CIN₂O | 325.1103 | 0.4 | 12.48 | +H |
| Quetiapine | [symbol] | C₂₁H₂₅N₃O₂S | 384.1739 | −0.4 | 5.38 | +H |
| Reboxetine | [symbol] | C₁₉H₂₃NO₃ | 314.1751 | 0.1 | 6.44 | +H |
| Ropivacaine | [symbol] | C₁₇H₂₆N₂O | 275.2121 | 1.2 | 3.23 | +H |
| Sertraline | [symbol] | C₁₇H₁₇C₁₂N | 306.0808 | −0.9 | 8.78 | +H |
| Tandospirone | [symbol] | C₂₁H₂₉N₅O₂ | 384.2392 | −0.5 | 3.63 | +H |
| Trazodone | [symbol] | C₁₉H₂₂CIN₅O | 372.1587 | 0.4 | 4.58 | +H |
| Venlafaxine | [symbol] | C₁₇H₂₇NO₂ | 278.2116 | 0.6 | 4.29 | +H |

### (8) Quantitative Analysis of Antidepressants

A quantitative analysis method based on the external standard method was established in UNIFI, and the default concentration levels of a standard curve were set in the list of target components for the qualitative method, which specifically are 1, 5, 10, 20, 50, 100 and 200 ng/g (indicating the concentration of each component) mixed standard working solutions and with a linear fitting mode for the standard curve, the MS^{E} data of mixed standard working solutions of antidepressants of a series of above concentrations of 1-200 mg/g were acquired; the MS^{E} data were imported into the UNIFI, analysis was established with the established analysis method; after the data analysis was completed, a standard working curve was automatically provided by software; the MS^{E} data of an actual environmental sludge sample were added to the quantitative analysis of the standards; and analysis was conducted to subsequently obtain contents of the target antidepressants in the sample.

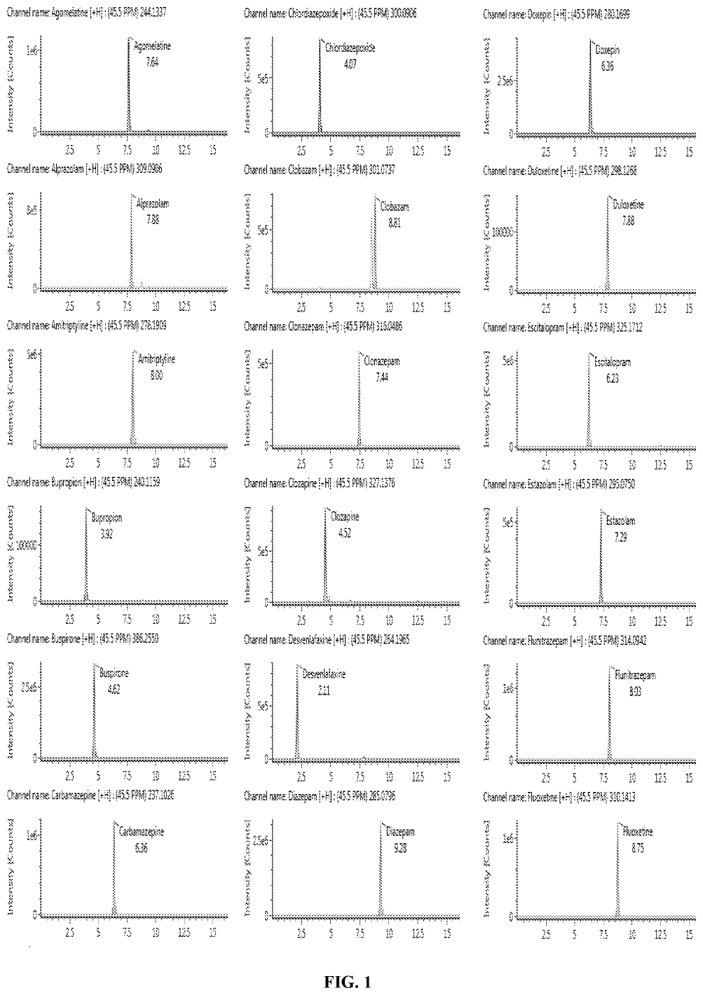

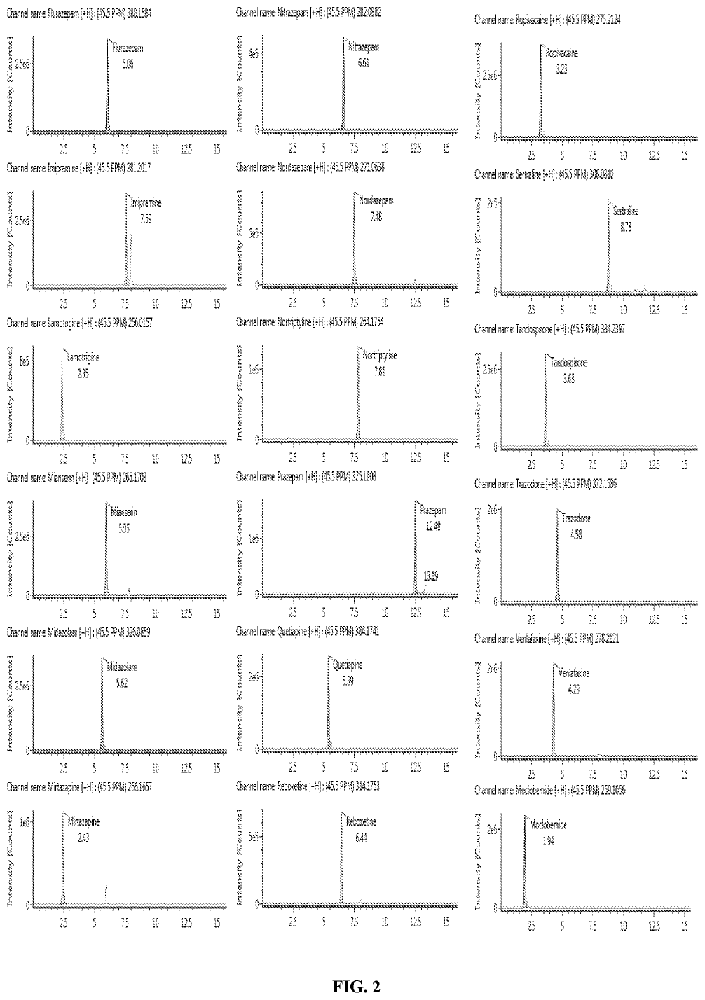

The MS^{E} data of individual compounds in the 100 g/L mixed standards of 36 target antidepressants were collected by using the UPLC/QTOF-MS sample detection method in step (6). The extracted ion chromatogram from the 36 target antidepressants are shown in FIG. 1 and FIG. 2.

Instruments as well as reagents and solutions used in the above process are prepared as follows:

Instruments: an ultra-performance liquid chromatography-tandem quadrupole time-of-flight mass spectrometer (Xevo QTOF MS, Waters, U.S.A.), ACQUITY UPLC BEH C18 columns (2.1 mm×100 mm, 1.7 μm, Waters, U.S.A.), a benchtop high-speed multifunction centrifuge (Centrifuge 5910 Ri, Eppendorf, Germany), an automatic concentrator (Auto EVA-60, Reeko, U.S.A.), pass-through purification cartridges (EMR-LPD with Carbon S, 6 mL, Agilent, U.S.A.), Milli-Q ultrapure water systems (Synergy, France), a DGJ-10C vacuum freeze dryer (Shanghai Bodeng Biological Science and Technology Co., Ltd.), a CYCQ-24D 24-well solid-phase extraction device (Hangzhou Chuanyi Electronic Co., Ltd.), an NMSG-12 multi-tube vortex mixer (Taizhou NuoMi Medical Technology Co., Ltd.), and an SB-800 DTD ultrasonic water bath (Ningbo Scientz Biotechnology Co., Ltd.).

Reagents: lamotrigine standards, purchased from TCI, Japan; desvenlafaxine, nortriptyline, amitriptyline and mianserin standards, purchased from GLPBIO, U.S.A.; alprazolam, estazolam, diazepam, flurazepam, flunitrazepam, chlordiazepoxide, clozapine, clonazepam, midazolam, prazepam, nordazepam, nitrazepam and clobazam standards, purchased from Cerilliant, U.S.A.; quetiapine, moclobemide, imipramine, mirtazapine, doxepin, carbamazepine, duloxetine, venlafaxine and sertraline standards, purchased from Shanghai Macklin Biochemical Co., Ltd.; ropivacaine, tandospirone, escitalopram, buspirone, agomelatine and bupropion standards, purchased from Meryer (Shanghai) Biochemical Technology Co., Ltd.; reboxetine standards, purchased from Shanghai Yuanye Bio-Technology Co., Ltd.; and trazodone and fluoxetine standards, purchased from Shanghai Aladdin Biochemical Technology Co., Ltd. Methanol, ethyl acetate, n-hexane, and HPLC-grade 25% ammonium hydroxide were HPLC-grade, and purchased from Shanghai Macklin Biochemical Co., Ltd. The ultrapure water used in the experiment (with the conductivity of 18.2 MQ) was prepared by the Milli-Q systems.

Standard stock solution: 0.01000 g of an antidepressant standard was accurately weighed, and then diluted with methanol to 10 mL in a 10 mL brown volumetric flask to prepare a standard stock solution with a concentration of 1000 mg/L, which was then preserved at −20° C. in the dark.

Standard mixed solution: 100 μL of the standard stock solution of each of the 36 antidepressants was placed in a 10 mL brown volumetric flask, and diluted with methanol to prepare a standard mixed solution with a concentration of 10 mg/L for each of the antidepressants. The working solutions of the mixed standards were prepared on site by progressively diluting the standard mixed solutions with methanol. The concentration in the standard mixed solutions or mixed standards in the present invention refers to the concentration of each individual component.

### Embodiment 2

This embodiment is intended to characterize the accuracy and precision of the method of the present invention, and to show the limit of detection of the method.

The target antidepressants were quantified by the external standard method. The sediment from a drinking water source was repeatedly washed with methanol; after the methanol was completely volatilized, the sediment was freeze-dried and used as blank sludge. The standard mixed solutions of target antidepressants (refer to the preparation of the standard mixed solution in Embodiment 1) are added to the blank sludge to the concentrations of 1, 5, 10, 20, 50, 100, and 200 ng/g (dw), respectively. The resulting sludge was recovered by the method developed in Embodiment 1 for matrix-spiked standard curves for quantification. The samples were loaded in turn to acquire data, which were then imported into UNIFI for drawing of the matrix-spiked standard curves. Meanwhile, the blank sludge was treated into sludge containing 100 ng/g (dw) standard target substances for quality control, and the content of the target antidepressant drug was measured by the above method to thus calculate a recovery rate (the computational formula of recovery rate: recovery rate=(A/B)×100%, where a substance to be measured with a known concentration A was added, and its concentration value B was determined by this method to obtain the recovery rate=(A/B)×100%)). In addition, a limit of quantification was determined with a signal-to-noise ratio of 3:1, and a limit of quantification was determined with a signal-to-noise ratio of 10:1, with the results shown in Table 2.

TABLE 2
Recovery rate, relative standard deviation, limit of detection
and limit of quantification of target antidepressants

| | Recovery | | Limit of | Limit of |
|---|---|---|---|---|
| | rate | RSD | detection | quantification |
| Compound | (%) | (%, n = 3) | (ng/g) | (ng/g) |
| Agomelatine | 66.19 | 0.74 | 0.74 | 2.47 |
| Alprazolam | 93.85 | 2.59 | 0.44 | 1.48 |
| Amitriptyline | 111.89 | 2.48 | 0.18 | 0.61 |
| Bupropion | 82.12 | 3.25 | 5.78 | 19.25 |
| Buspirone | 88.48 | 1.04 | 0.34 | 1.12 |
| Carbamazepine | 78.50 | 2.70 | 0.89 | 2.98 |
| Chlordiazepoxide | 82.28 | 1.77 | 1.07 | 3.58 |
| Clobazam | 77.75 | 1.76 | 1.46 | 4.87 |
| Clonazepam | 113.71 | 0.42 | 0.55 | 1.85 |
| Clozapine | 66.41 | 0.80 | 0.70 | 2.32 |
| Desvenlafaxine | 78.53 | 2.27 | 0.66 | 2.21 |
| Diazepam | 102.40 | 0.42 | 0.35 | 1.16 |
| Doxepin | 101.59 | 1.23 | 3.96 | 13.21 |
| Duloxetine | 63.13 | 0.04 | 13.47 | 44.91 |
| Escitalopram | 92.52 | 1.08 | 0.18 | 0.59 |
| Estazolam | 88.17 | 1.42 | 1.26 | 4.21 |
| Flunitrazepam | 114.26 | 4.09 | 0.47 | 1.56 |
| Fluoxetine | 86.83 | 2.65 | 0.88 | 2.94 |
| Flurazepam | 84.42 | 1.48 | 0.24 | 0.82 |
| Imipramine | 88.18 | 1.48 | 0.37 | 1.22 |
| Lamotrigine | 78.39 | 3.00 | 3.71 | 12.38 |
| Mianserin | 89.18 | 0.48 | 10.74 | 35.81 |
| Midazolam | 81.50 | 1.45 | 0.14 | 0.47 |
| Mirtazapine | 72.26 | 1.40 | 3.47 | 11.58 |
| Moclobemide | 91.58 | 0.29 | 1.90 | 6.33 |
| Nitrazepam | 91.89 | 1.87 | 0.31 | 1.05 |
| Nordazepam | 115.66 | 2.98 | 0.63 | 2.08 |
| Nortriptyline | 102.77 | 1.76 | 0.41 | 1.38 |
| Prazepam | 102.72 | 0.61 | 1.16 | 3.87 |
| Quetiapine | 80.79 | 1.40 | 0.17 | 0.56 |
| Reboxetine | 96.71 | 0.28 | 3.55 | 11.84 |
| Ropivacaine | 96.61 | 3.43 | 0.24 | 0.80 |
| Sertraline | 75.85 | 2.67 | 2.29 | 7.65 |
| Tandospirone | 86.21 | 1.60 | 0.18 | 0.61 |
| Trazodone | 86.26 | 1.33 | 0.27 | 0.89 |
| Venlafaxine | 96.14 | 2.06 | 0.44 | 1.46 |

### Embodiment 3

This embodiment is intended to embody the application effects of the method of the present invention in actual sludge samples. Sludge 1 and Sludge 2 are activated sludge from different WWTPs, and Sludge 3 is the sediment from a black and odorous river in Guangzhou City. The detection of the 36 target antidepressants by the method according to Embodiment 1 is shown in Table 3, with N.D. indicating not detected and <LOQ indicating detected but below the limit of quantification of this method.

TABLE 3
Detection of 36 target antidepressants in actual sludge samples

| | Residue of antidepressants in | | |
|---|---|---|---|
| | environmental sample (ng/g) | | |
| Compound | Sludge 1 | Sludge 2 | Sludge 3 |
| Agomelatine | N.D. | N.D. | N.D. |
| Alprazolam | N.D. | N.D. | N.D. |
| Amitriptyline | N.D. | N.D. | N.D. |
| Bupropion | 27.69 | N.D. | N.D. |
| Buspirone | 6.05 | 6.14 | 6.20 |
| Carbamazepine | 4.26 | N.D. | N.D. |
| Chlordiazepoxide | N.D. | N.D. | N.D. |
| Clobazam | N.D. | N.D. | N.D. |
| Clonazepam | N.D. | N.D. | N.D. |
| Clozapine | N.D. | 2.69 | 6.51 |
| Desvenlafaxine | N.D. | N.D. | N.D. |
| Diazepam | 6.00 | 6.13 | N.D. |
| Doxepin | N.D. | 3.71 | 3.74 |
| Duloxetine | N.D. | <LOQ | N.D. |
| Escitalopram | N.D. | N.D. | N.D. |
| Estazolam | N.D. | 17.87 | N.D. |
| Flunitrazepam | N.D. | N.D. | N.D. |
| Fluoxetine | 11.40 | N.D. | N.D. |
| Flurazepam | N.D. | N.D. | N.D. |
| Imipramine | N.D. | N.D. | N.D. |
| Lamotrigine | N.D. | N.D. | N.D. |
| Mianserin | N.D. | N.D. | 6.16 |
| Midazolam | N.D. | N.D. | N.D. |
| Mirtazapine | N.D. | 11.11 | N.D. |
| Moclobemide | N.D. | N.D. | N.D. |
| Nitrazepam | N.D. | N.D. | N.D. |
| Nordazepam | 8.15 | N.D. | N.D. |
| Nortriptyline | N.D. | N.D. | N.D. |
| Prazepam | N.D. | N.D. | N.D. |
| Quetiapine | N.D. | N.D. | N.D. |
| Reboxetine | 16.06 | N.D. | N.D. |
| Ropivacaine | N.D. | N.D. | N.D. |
| Sertraline | N.D. | 10.14 | 10.87 |
| Tandospirone | N.D. | N.D. | N.D. |
| Trazodone | N.D. | N.D. | N.D. |
| Venlafaxine | N.D. | 7.71 | N.D. |

What is claimed is:

1. A method for simultaneous and efficient detection of residues of 36 antidepressants in sludge, comprising the following steps:
step 1, extraction of target drugs: weighing a sludge sample, and then performing solvent extraction to obtain an extraction solution containing the antidepressants;
step 2, purification of the extraction solution: purifying the extraction solution;
step 3, detection of the purified extraction solution by a technique of ultra-performance liquid chromatography-tandem mass spectrometry; and
step 4: qualitative and quantitative analysis of the antidepressants;
wherein the 36 antidepressants comprise agomelatine, alprazolam, amitriptyline, bupropion, buspirone, carbamazepine, chlordiazepoxide, clobazam, clonazepam, clozapine, desvenlafaxine, diazepam, doxepin, duloxetine, escitalopram, estazolam, flunitrazepam, fluoxetine, flurazepam, imipramine, lamotrigine, mianserin, midazolam, mirtazapine, moclobemide, nitrazepam, nordazepam, nortriptyline, prazepam, quetiapine, reboxetine, ropivacaine, sertraline, tandospirone, trazodone, and venlafaxine;
the step of performing solvent extraction to obtain the extraction solution containing the antidepressants comprises the following steps: freeze-drying the sludge sample, grinding and sieving the freeze-dried sludge, weighing the sludge sample, successively adding an inorganic extractant and an organic extractant to the sludge sample, mixing a resultant evenly, and then performing ultrasonic and centrifugal extraction to collect a supernatant, wherein the inorganic extractant is an aqueous ammonia solution, and the organic extractant is one or more of methanol, ethyl acetate and n-hexane; and
the step of purifying the extraction solution comprises: passing the extraction solution collected in step 1 through pass-through EMR-LPD cartridges containing Carbon S, collecting a purified effluent, drying the purified effluent with nitrogen at room temperature, redissolving the dried effluent in methanol, mixing a resultant evenly, and filtering a resulting mixture to collect a filtrate, wherein the filtrate is preserved in the dark for later on-instrument detection.

2. The method according to claim 1, wherein detection conditions for the technique of ultra-performance liquid chromatography-tandem mass spectrometry comprise:
conditions for liquid chromatography: ACQUITY UPLC BEH C18 columns, 2.1 mm×100 mm, column temperature: 40° C., flow rate: 0.35 mL/min, injection volume: 2 μL, mobile phase A: 0.1% formic acid in Milli-Q water, and mobile phase B: acetonitrile; and gradient elution conditions: 0-28 min, 90/6-10% of A; 28-30 min, 10/6-90% of A, for 2 min, with the mobile phases A and B in an eluent being 100% in total in each stage; and
detection conditions for mass spectrometry: with an electrospray ion source and under a positive ion mode, simultaneous acquisition of MS data of high- and low-collision energy by using an MS^{E} scanning mode in a single injection, wherein compound precursor ions are collected in low-energy channels and fragment ions are collected in high-energy channels; collision voltage: low energy: 6 V, high energy: 10 V-40 V; scanning range: m/z: 50-1000, ion source temperature: 120° C.; capillary voltage: 2.5 kV; cone voltage 40 V; cone gas flow rate: 50 L/Hr; and drying gas: nitrogen, temperature: 500° C., and flow rate: 600 L/Hr.

3. The method according to claim 1, wherein the step of performing the solvent extraction to obtain the extraction solution containing the antidepressants comprises the following steps:
(1) freeze-drying: freeze-drying the sludge sample for 48 hours by using a freeze dryer at −60° C. under vacuum conditions, grinding the freeze-dried sludge sample, sieving the ground sludge sample through a 100-mesh sieve to obtain homogeneous sludge particles, and cold-preserving the sludge particles at −20° C.; and
(2) ultrasonic and centrifugal extraction: weighing 0.5 g of the sludge sample, placing the sludge sample in a 50 mL centrifuge tube, and performing the following steps:
step A: adding 4 mL of an aqueous ammonia solution, which is adjusted to pH 11 with HPLC-grade 25% ammonium hydroxide, and 1 mL of methanol to a sample tube, and vortexing the sample for 5 min;
step B: adding 5 mL of ethyl acetate to the sample tube, performing vortexing for thorough mixing, then placing the sample tube in an ultrasonic bath for extracting for 10 min, then centrifuging for 5 min at 4500 rpm to collect a supernatant, repeating the steps of ultrasonication and centrifugation once, and combining the supernatants; and
step C: adding 4 mL of n-hexane to the sample tube, performing vortexing for thorough mixing, then placing the sample tube in the ultrasonic bath for extracting for 10 min, then centrifuging for 5 min at 4500 rpm, collecting a supernatant and combining the supernatant to obtain the extraction solution.

4. The method according to claim 1, wherein the qualitative and quantitative analysis of the antidepressants comprises: performing qualitative analysis by the exact mass, retention time, and characteristic ions; and performing quantitative analysis by an external standard method.

5. The method according to claim 4, wherein the qualitative and quantitative analysis comprises:
a qualitative method, comprising: with qualitative analysis on target drugs performed in a UNIFI scientific information system, before the qualitative analysis, drawing chemical structures of target antidepressants to construct a target drug database, wherein the target drug database is imported into the UNIFI scientific information system; then, establishing an analysis method, wherein after a desired ionization mode is selected, the target drug database is imported as a list of target components for the analysis method, and corresponding screening parameters are set, specifically with an exact mass error of less than 3 ppm for the compound precursor ions, and with a exact mass error of less than 10 ppm for the fragment ions; acquiring MS^{E} data of individual target antidepressant standards at 100 μg/L by using a detection method in step 3, then analyzing the data in the UNIFI scientific information system, and performing matching one by one to obtain measured exact masses under an instrument method, retention time, and mass spectrum information under high- and low-energy for the individual antidepressants; and assigning the information obtained by using the standards to the individual antidepressants in the database, and establishing a qualitative method for the sample by reusing the database containing the measured exact mass, retention time, and fragment ion information of the individual antidepressants, wherein a matching principle for the target antidepressants in an actual sample is as follows: desired fragment ions are matched with the exact mass error of less than 2 ppm and a retention time error of less than 0.1 min; and
a quantitative method, comprising: establishing a quantitative analysis method based on the external standard method in UNIFI, and setting default concentration levels of a standard curve in the list of target components for the qualitative method, wherein the default concentration levels are 1, 5, 10, 20, 50, 100 and 200 ng/g mixed standard working solutions and with a linear fitting mode for the standard curve; acquiring MS^{E} data of mixed standard working solutions of antidepressants with a series of concentrations, importing the MS^{E} data into the UNIFI, establishing analysis with the established analysis method, and after the data analysis is completed, automatically providing a standard working curve by software, adding MS^{E} data of an actual environmental sludge sample to the quantitative analysis of the standards, and performing analysis to subsequently obtain concentrations of the target antidepressants in the sample.

6. The method according to claim 1, wherein the method is used in detection of residues of 36 antidepressants in sludge, wherein the 36 antidepressants comprise: agomelatine, alprazolam, amitriptyline, bupropion, buspirone, carbamazepine, chlordiazepoxide, clobazam, clonazepam, clozapine, desvenlafaxine, diazepam, doxepin, duloxetine, escitalopram, estazolam, flunitrazepam, fluoxetine, flurazepam, imipramine, lamotrigine, mianserin, midazolam, mirtazapine, moclobemide, nitrazepam, nordazepam, nortriptyline, prazepam, quetiapine, reboxetine, ropivacaine, sertraline, tandospirone, trazodone, and venlafaxine.

7. The method according to claim 2, wherein the step of performing the solvent extraction to obtain the extraction solution containing the antidepressants comprises the following steps:
(1) freeze-drying: freeze-drying the sludge sample for 48 hours by using a freeze dryer at −60° C. under vacuum conditions, grinding the freeze-dried sludge sample, sieving the ground sludge sample through a 100-mesh sieve to obtain homogeneous sludge particles, and cold-preserving the sludge particles at −20° C.; and
(2) ultrasonic and centrifugal extraction: weighing 0.5 g of the sludge sample, placing the sludge sample in a 50 mL centrifuge tube, and performing the following steps:
step A: adding 4 mL of an aqueous ammonia solution, which is adjusted to pH 11 with HPLC-grade 25% ammonium hydroxide, and 1 mL of methanol to a sample tube, and vortexing the sample for 5 min;
step B: adding 5 mL of ethyl acetate to the sample tube, performing vortexing for thorough mixing, then placing the sample tube in an ultrasonic bath for extracting for 10 min, then centrifuging for 5 min at 4500 rpm to collect a supernatant, repeating the steps of ultrasonication and centrifugation once, and combining the supernatants; and
step C: adding 4 mL of n-hexane to the sample tube, performing vortexing for thorough mixing, then placing the sample tube in the ultrasonic bath for extracting for 10 min, then centrifuging for 5 min at 4500 rpm, collecting a supernatant and combining the supernatant to obtain the extraction solution.

8. The method according to claim 2, wherein the qualitative and quantitative analysis of the antidepressants comprises: performing qualitative analysis by the exact mass, retention time, and characteristic ions; and performing quantitative analysis by an external standard method.

9. The method according to claim 8, wherein the qualitative and quantitative analysis comprises:
a qualitative method, comprising: with qualitative analysis on target drugs performed in a UNIFI scientific information system, before the qualitative analysis, drawing chemical structures of target antidepressants to construct a target drug database, wherein the target drug database is imported into the UNIFI scientific information system; then, establishing an analysis method, wherein after a desired ionization mode is selected, the target drug database is imported as a list of target components for the analysis method, and corresponding screening parameters are set, specifically with an exact mass error of less than 3 ppm for the compound precursor ions, and with a exact mass error of less than 10 ppm for the fragment ions; acquiring MS^{E} data of individual target antidepressant standards at 100 μg/L by using a detection method in step 3, then analyzing the data in the UNIFI scientific information system, and performing matching one by one to obtain measured exact masses under an instrument method, retention time, and mass spectrum information under high- and low-energy for the individual antidepressants; and assigning the information obtained by using the standards to the individual antidepressants in the database, and establishing a qualitative method for the sample by reusing the database containing the measured exact mass, retention time, and fragment ion information of the individual antidepressants, wherein a matching principle for the target antidepressants in an actual sample is as follows: desired fragment ions are matched with the exact mass error of less than 2 ppm and a retention time error of less than 0.1 min; and
a quantitative method, comprising: establishing a quantitative analysis method based on the external standard method in UNIFI, and setting default concentration levels of a standard curve in the list of target components for the qualitative method, wherein the default concentration levels are 1, 5, 10, 20, 50, 100 and 200 ng/g mixed standard working solutions and with a linear fitting mode for the standard curve; acquiring MS^{E} data of mixed standard working solutions of antidepressants with a series of concentrations, importing the MS^{E} data into the UNIFI, establishing analysis with the established analysis method, and after the data analysis is completed, automatically providing a standard working curve by software, adding MS^{E} data of an actual environmental sludge sample to the quantitative analysis of the standards, and performing analysis to subsequently obtain concentrations of the target antidepressants in the sample.
